(19) United States
(12) Patent Application Publication
(10) Pub. No.: US 2007/0261155 A1
(43) Pub. Date: Nov. 15, 2007

(54) Mask-Form Goggles, Particularly for Sporting Use

(75) Inventor: Vittorio Tabacchi, Pieve Di Cadore (BL) (IT)

Correspondence Address: RATNERPRESTIA, P O BOX 980, VALLEY FORGE, PA, 19482-0980, US

(73) Assignee: SAFILO SOCIETA AZIONARIA FABBRICA ITALIANA LAVORAZIONE OCCHIALI S.P.A., PIEVE DI CADORE (BL) (IT)

(21) Appl. No.: 11/663,259

(22) PCT Filed: Sep. 29, 2004
(86) PCT No.: PCT/IT04/00537

§ 371 Date: Mar. 19, 2007

(51) Int. Cl. A61F 9/02

(52) U.S. Cl. 2/439

(57) ABSTRACT

Mask-form goggles, particularly for sporting use, are described which include a first front mount and a second juxtaposed mount and a lens held on at least one of the mounts. The first and the second mounts extend in such a manner that they surround the lens, and a releasable mutual coupling device is provided between the first and the second mount and is such as to permit access to the inside of the mask, when it is being worn, by moving one of the mounts away from the other.

This application is a U.S. National Phase Application of PCT International Application PCT/IT2004/000537 which is incorporated by reference herein.

### TECHNICAL FIELD

The present invention relates to mask-form goggles, and in particular to goggles for sporting use.

### TECHNOLOGICAL BACKGROUND

The invention is applicable in particular, although not exclusively, in the specific technical field of goggles of the single-lens mask type arranged to be fitted and held on the face in order to protect the eyes during sporting activity, in particular in the practice of skiing, snowboarding and other similar sporting disciplines.

In this field, for example, mask-form goggles provided with a frame for holding an interchangeable lens are known. In known applications of this type it is provided that the frame comprises a mount having a portion associated with a seal of spongy material suitable for ensuring sealed coupling of the frame to the user's face, and a second portion which extends from the first mount portion and which is arranged to hold the lens, for example, in a releasable manner, in order to ensure the interchangeability thereof for the purpose of replacing the lens in the case of damage, or for the purpose of replacing it by other lenses of a different colour, or more simply for the purpose of facilitating the operations of cleaning the lens after removing it from the lens-carrying mount.

### BRIEF DESCRIPTION OF THE INVENTION

In this specific field, a principal object of the invention is to provide mask-form goggles that are designed structurally and functionally to obtain improved convenience of use, in particular in order to permit free and complete access, when the mask is being worn, to the inside of the mask at the location of the front region of the face protected by the lens in order, for example, to put on or clean any spectacles or in order to adjust the position thereof on the face, without having to loosen or remove the mask from the head.

Another object is to improve the transpiration of the user's face, opposing the misting-up of the lens and facilitating the operations of cleaning it.

Yet another object is to permit rapid replacement of the lens in the mask.

Yet another object is to render interchangeable, in addition to the lens, at least part of the frame in order to obtain in the same mask frame combinations of different colours combinable with as many different colours of interchangeable lens, for a more distinct personalisation of the mask which satisfies the most varied requirements and tastes of the users.

Those and other objects which will emerge clearly hereinafter are achieved by mask-form goggles which comprise a first front mount and a second juxtaposed mount and a lens held on at least one of the mounts. The first and the second mounts extend in such a manner that they surround the lens, and means for releasable mutual coupling are provided between the first and the second mount and are such as to permit access to the inside of the mask, when it is being worn, by moving one of the mounts away from the other.

### BRIEF DESCRIPTION OF THE DRAWINGS

Further features and advantages of the invention will become clearer from the following detailed description of some of its preferred embodiments which are illustrated by way of non-limiting example with reference to the appended drawings in which:

FIG. 1 is a perspective front elevational view, with disassembled parts, of a first embodiment of mask-form goggles according to the invention,

FIG. 2 is a front elevational view of a detail of the mask of FIG. 1,

FIGS. 3 and 4 are partly sectioned views on an enlarged scale of respective details of the mask of the preceding Figures,

FIG. 4A is a sectioned partial view on an enlarged scale of a variant of a detail of the mask of the preceding Figures,

FIG. 5 is a top view of the mask of the preceding Figures in a partially assembled condition,

FIG. 6 is a plan view of a further detail of the mask of the preceding Figures,

FIG. 7 is a perspective front elevational view, with disassembled parts, of a second embodiment of the invention,

FIG. 8 is a partly sectioned perspective view on an enlarged scale of a detail of the mask of FIG. 7,

FIGS. 9 and 10 are side elevational views of the mask-form goggles of FIG. 7 in two different operative conditions,

FIGS. 11 and 12 are side elevational views of a detail of the mask-form goggles of FIG. 7 in two variants,

FIG. 13 is a perspective front elevational view of a third embodiment of the invention,

FIG. 14 is a partly sectioned view on an enlarged scale of a detail of the mask-form goggles of FIG. 13,

FIG. 15 is a perspective front elevational view of a fourth embodiment of the invention,

FIG. 16 is a partly sectioned view on an enlarged scale of a detail of the mask-form goggles of FIG. 15,

FIG. 17 is a perspective partly sectioned view of a fifth embodiment of the invention,

FIG. 18 is a perspective view of the mask-form goggles of FIG. 17,

FIG. 19 is a partly sectioned perspective view of a detail of the mask-form goggles of FIG. 17,

FIGS. 20 and 21 are plan views of the mask-form goggles of FIG. 17 in two different configurations.

### PREFERRED EMBODIMENTS OF THE INVENTION

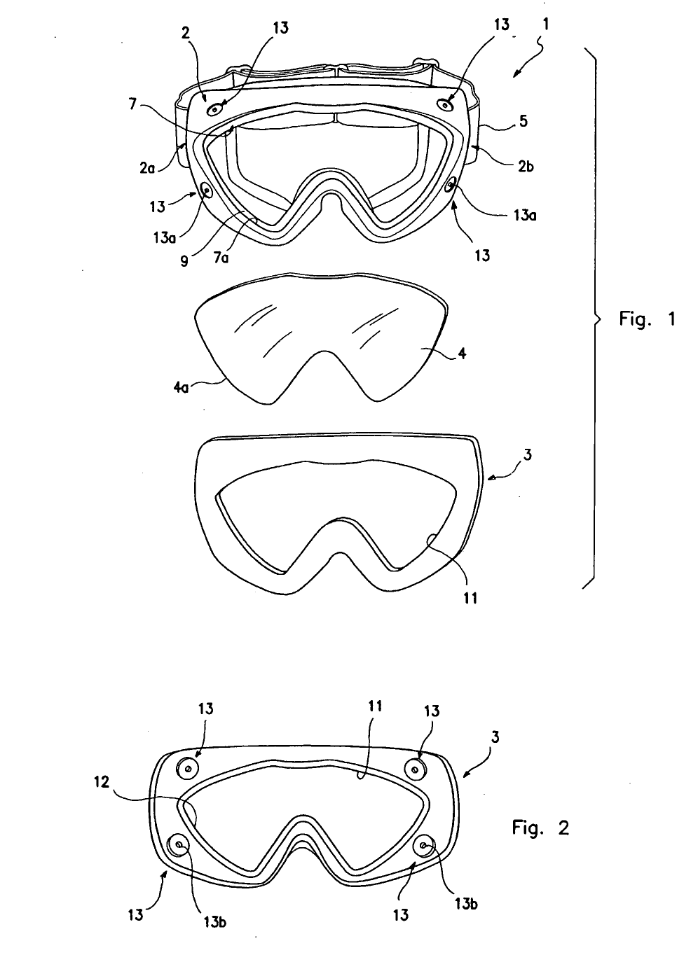

Referring first of all to FIGS. 1 to 6, a first embodiment of mask-form goggles produced in accordance with the present invention is generally indicated 1.

The goggles 1 are produced in the form of a single-lens mask and comprise a frame having a first mount and a second mount indicated 2 and 3, respectively, which are arranged to hold a lens 4 and which are preferably produced in a suitable plastics material.

The lens 4 may be produced as a single structure (single-lens) or as a double lens. 5 indicates a resilient strap with adjustable tensioning for holding the mask on the user's head, having a structure which is conventional per se and which is constrained on the first mount 2 at the location of its opposite lateral ends 2a, 2b. The first mount 2 will be identified hereinafter also as the “internal” mount of the mask because it faces the user's face, when the mask is being worn, while the mount 3, on the other hand, will be referred to as the “external” mount because it is coupled to the mount 2 on the side remote from the user's face.

The internal mount 2 is provided with a sealing element 6 extending along a peripheral edge of the mount and capable of coming into contact with the face when the mask is being worn. The sealing element 6 is produced in a soft material (for example rubber) or a spongy material, having absorbent and transpiring properties (for example in foam rubber) and acts as a soft interface structure interposed between the internal mount and the face, for the purpose of ensuring correct sealed coupling of the mask to the face in order to prevent the ingress of water, dust, snow or other undesirable agents into the mask and at the same time to permit sufficient transpiration.

The internal mount 2 also comprises a front through-opening 7 whose peripheral edge 7a has a profile with a configuration corresponding substantially to the peripheral profile 4a of the lens 4. At the location of the opening 7, a groove 8 which extends along the entire profile 7a and which constitutes an at least partial engagement seat for a bezel element 9 is provided in the internal mount. The bezel element has a general closed-contour configuration, as illustrated in FIG. 6, with a shape corresponding to that of the opening 7 and is advantageously produced in plastics material, for example reinforced with glass fibres, or in metal material.

The bezel 9 is constrained in the groove 8 by the interlocking of notches 9b provided on the edge of the bezel (FIG. 6) and corresponding appendages (not shown) provided inside the groove 8. The notches and the associated appendages of the groove are provided in a variable position along the entire periphery of the bezel.

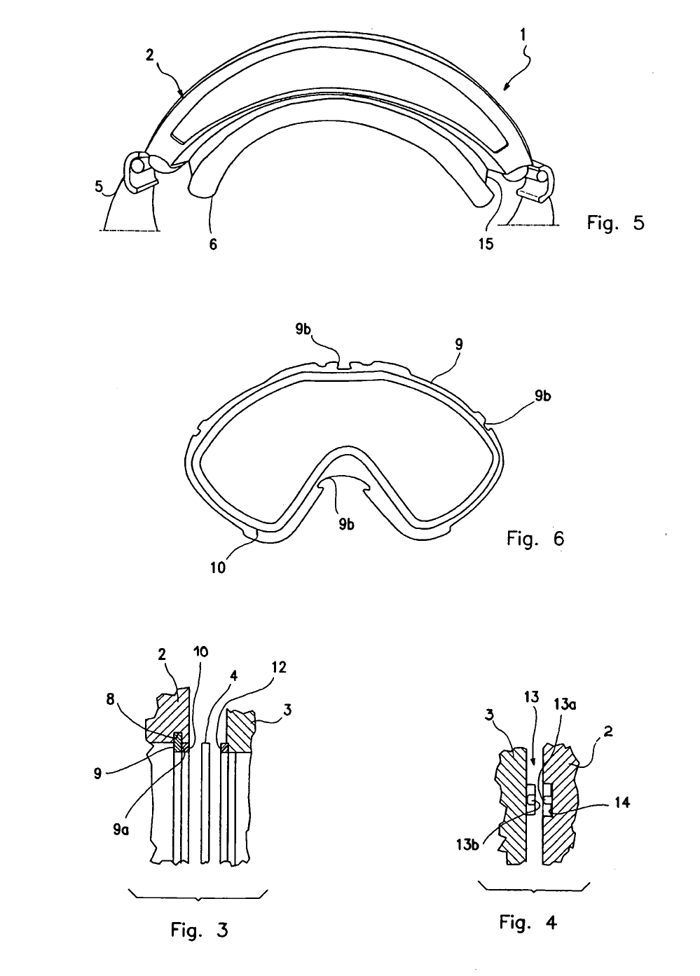

The bezel element 9 has a cross-section with a first portion accommodated and held in the groove 8 and a second portion, projecting into the opening 7, on which a locating surface 9a for the lens 4 is identified. Preferably, a seal 10 of soft material (for example rubber or of spongy material or another material having similar characteristics) is fitted on the locating surface 9a and the lens 4 bears on it when the mask has been assembled, as shown in FIG. 3. It will be appreciated that the seal 10 is selected to be of a size and cross-section such as to conceal the bezel 9 completely from view when the external mount is separated from the internal mount.

The external mount 3 is in turn provided with a front through-opening 11 whose peripheral configuration is produced in a manner corresponding substantially to the peripheral profile of the lens 4 but has dimensions such that, after the coupling of the external mount to the internal mount, as will be explained in detail hereinafter, the lens 4 is held firmly in the mask. The external mount 3 is also advantageously provided with a soft seal 12 (of material corresponding to that with which the seal 10 is produced) which is fitted onto the surface of the external mount facing the internal mount, along the peripheral profile of the opening 11, so that it is in a position facing the seal 10. The lens 4 is thus held between the mounts, after they have been coupled to each other, in bearing contact on the seals 10 and 12, thus producing a sealed coupling between the mounts.

For the releasable coupling of the external mount 3 to the internal mount 2, means and counter-means for snap-locking simply by applying pressure are preferably provided on the two mounts, respectively, and can be engaged with each other in order to secure the internal mount releasably to the external mount, with the lens consequently being held in position.

The means and counter-means for releasable locking comprise press-studs, preferably numbering four, which are all generally indicated 13. The studs are positioned between the front surfaces for coupling the mounts to each other in the preferred configuration illustrated in FIG. 2.

Preferably, the four studs are positioned, respectively, in the peripheral regions close to the four vertices of the external mount. However, various other configurations, both in terms of the number of studs provided and in terms of the positioning thereof relative to one another, are also possible.

The studs 13 may be produced integrally with the respective mounts by injection-moulding or they may be produced in metal material for subsequent fitting onto the mask. They may be circular, oblong, square, rectangular or the like, as desired. When produced in metal material, the studs 13 are preferably let partially or totally into the corresponding mount in order to limit or eliminate portions thereof projecting from the surface of the mounts. As shown in FIG. 4, at least the appendage 13a of the stud 13, which is capable of being snap-coupled in the opposite seat 13b of the stud, is accommodated in a suitable recess 14 in the mount 3 in such a manner that it does not project at all from the surface profile of the mount.

Likewise, if produced by injection-moulding, the studs 13 are constructed in such a manner as to limit or not to provide portions projecting from the profile of the corresponding mount.

As an alternative to the snap-studs, there is provision for the use of means constituted by appendages or protuberances projecting from the external mount and capable of coupling engagement in respective seats or cavities formed in the internal mount in a corresponding position. It is preferably provided that each protuberance engages a respective cavity formed in the internal mount, the cavity having a slot-form opening with a cross-section such as to ensure interlocking after the protuberance has been pressed into the cavity.

With particular reference to FIG. 4A, means comprising slides or push-buttons 16 which are accessible from the front side of the external mount and which are provided with a slide portion 16a extending through a respective cavity 17a formed in the external mount are provided as further alternative securing means to the studs. The portion 16a of each slide also extends into a respective cavity 17b formed in the internal mount, the cavity 17b being in a form such as to permit the interlocking of the slide by form-fitting with the internal end 16b of the slide, or, conversely, to permit release of the end of the slide, depending on the “open-closed” position of the slide/push button on the external mount.

The interlocking of the internal ends of the slides with the cavities in the internal mount may be promoted by the use of resiliently flexible materials, or by the use of any form of metal spring. 15 indicates a connecting structure between the main body of the internal mount and the soft seal 6 for contact with the user's face. This structure is produced in a material having a characteristic of greater flexibility compared with the portions to which it is connected and it extends along the entire peripheral edge of the mask, in a coupled arrangement with the seal 6. The characteristic of flexibility and extendability is conferred on the structure 15 either by using an appropriate material (differing from the other portions of the mask) or by adopting particular “bellows” or “accordion” shapes which are distinguished by a distinct compliance in predetermined directions, or by combining those two arrangements. The connecting structure 15 makes it possible to compensate for the possible resilient deformation induced in the internal mount by any states of tension generated in the coupling to the external mount. This deformation is absorbed by the structure 15 by its resilient compliance and it is therefore not transferred to the internal region of contact with the face in which the seal 6, by adapting itself to the most diverse morphologies of the face, remains firmly in contact therewith, thus ensuring that the mask is sealed.

In use, in order to assemble the mask, the user positions the lens 4 in the corresponding seat defined in the internal mount 2, bearing on the seal 10, and places the external mount 3 in corresponding superposition, locking it on the internal mount by closing the press-studs 13. Conversely, simply by opening the studs, the external mount can be uncoupled from the internal mount, thus permitting the removal of the lens and also complete accessibility to the internal region of the mask without, moreover, requiring the disengagement of the internal mount from the user's face.

The invention therefore enables the lens to be interchanged in a manner which is simple and quick for the user, thus permitting replacement of the lens in the case of damage, or making it possible to use lenses of different colours, or also permitting easy removal of the lens for cleaning operations.

The invention also has the advantage of ensuring that the external mount can be interchanged, thus enabling the user to replace that mount, in the same mask, by others having a different colour, also selecting the preferred combination with a specific lens. It is thus possible to provide groups of external mounts and lenses of different colours in order to make a range of combinations available to the user.

Yet another advantage resides in the fact that the ability to remove the external mount and the lens enables the exchange of air inside the mask to be increased, which brings about a consequent greater transpiration of the user's face, which is particularly important in the case of excessive sweating. This function is also ensured without requiring the user to take the mask off his face.

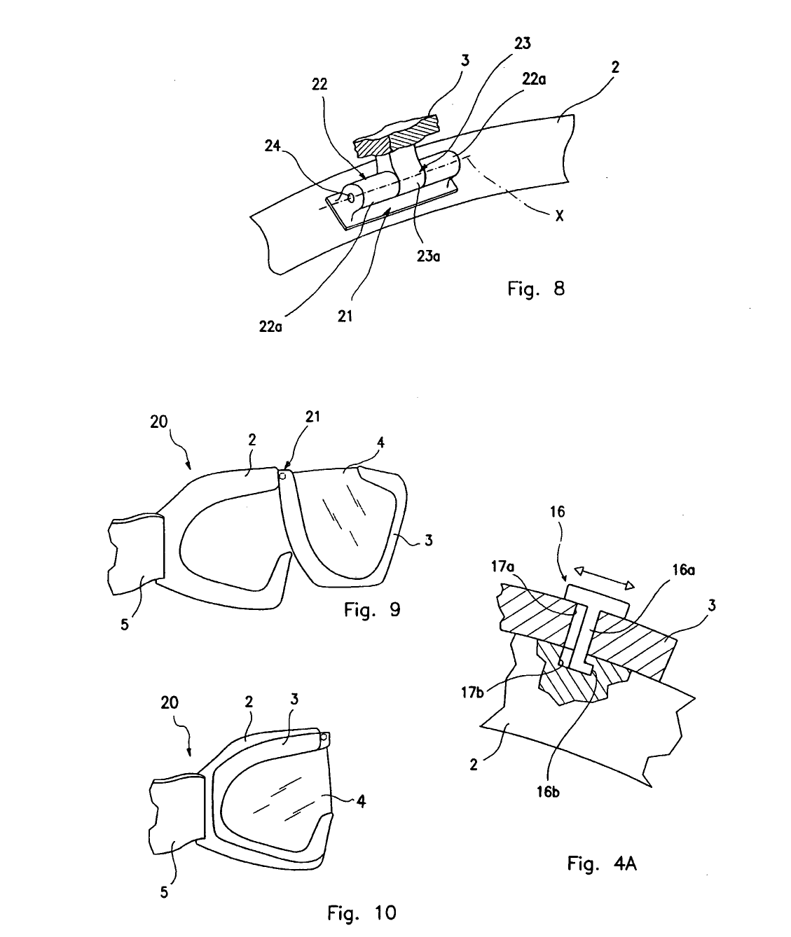

Referring to FIGS. 7 to 12, 20 indicates a second embodiment of mask-form goggles according to the invention in which components analogous to those of the previous embodiment are indicated with the same reference numerals.

The mask-form goggles 20 differ principally from those of the previous embodiment owing to the fact that they also provide a hinge device, generally indicated 21, between the internal mount 2 and the external mount 3, by means of which the mounts are articulated to each other in such a manner that they can pivot with respect to each other about a hinge axis indicated X in the drawings.

In more detail, the hinge device 21 is located in a central front portion of the mounts, in the region corresponding substantially to the eyebrow arch, as illustrated clearly in FIG. 7. The device comprises a first hinge element 22 and a second hinge element 23 which are associated with the internal mount and the external mount, respectively, and which are also rotatably coupled to each other about the hinge axis. In a preferred embodiment, the hinge elements 22, 23 comprise respective eyelet formations 22a, 23a which are coaxial with each other and which are coupled rotatably by means of a hinge pin 24. The eyelet formations 22a, 23a are produced in plastics material, for example by injection-moulding, and are optionally produced integrally with the corresponding mounts or, alternatively, they may be produced in a manner which is structurally independent of the mounts, preferably in metal material, and are arranged to be fitted to the respective mounts by screw securing means or riveted pin securing means, or by adhesive bonding or another mechanical securing system.

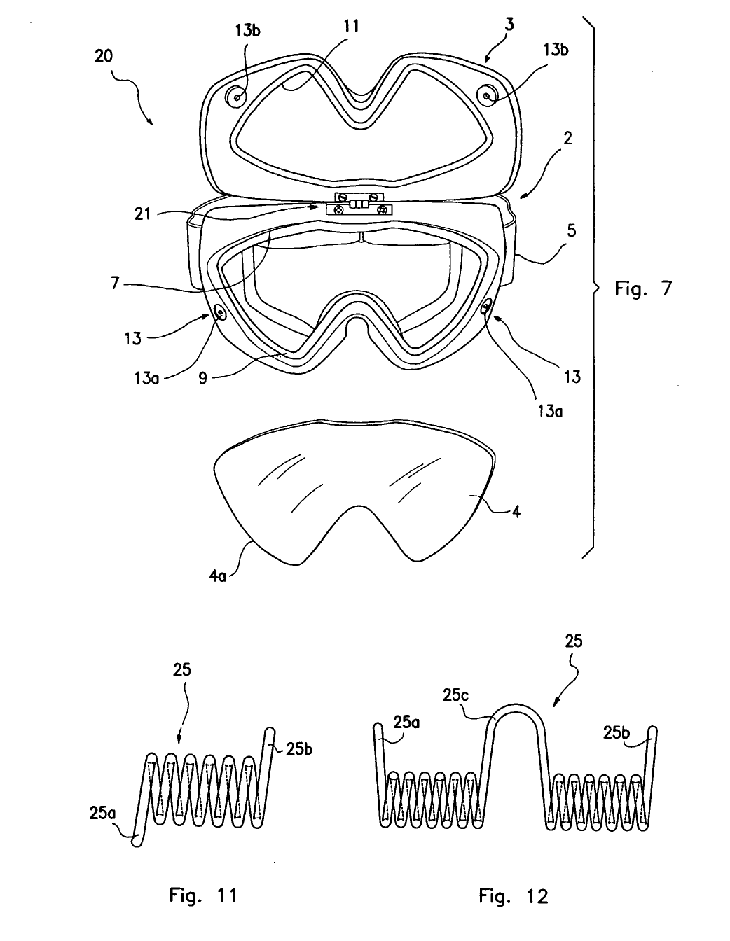

By means of the hinge device 21, the external mount 3 can be pivoted about the hinge axis X, between a first, closing, position with superposition on the internal mount 2 (FIG. 10) and a second, raised, opening position away from the internal mount (FIG. 9), this second position being such as to ensure complete access to the inside of the mask through the front opening 7 in the first mount, for example, in order to disengage the lens 4 and to enable it to be removed from the mask.

From the closed position of FIG. 10, the mounts can also be locked to each other by means of a pair of press-studs 13 arranged in reflective positions along the lower portions of the mounts, on the side frontally opposite the eyebrow arch region. The studs 13 are structurally and functionally identical to those provided in the previous embodiment. It is provided that the external mount 3 is pivotable manually or, alternatively, that it is resiliently urged towards the raised position away from the internal mount.

To that end, a spring 25 which is associated with the hinge elements 22, 23 and which acts between the mounts is provided. The spring 25 may be produced, for example, in the form of a cylindrical helical torsion spring which has opposite axial ends 25a, 25b secured to the internal mount and to the external mount, respectively, and which is also arranged coaxially with the hinge axis X (FIG. 11). Alternatively, the spring 25 may be in the form of a torsion spring with both of the axial ends 25a, 25b secured to the internal mount and with an intermediate portion 25c of a suitable shape secured to the external mount (FIG. 12). In both cases, the resilient urging of the spring is such as to return the external mount to the raised position when the press-studs 13 are open. Conversely, the external mount 3 can be pivoted towards the closed position, against the resilient force generated by the spring.

In order to urge the external mount automatically towards the raised position, a further alternative provides a system of springs of the “gas spring” type, that is to say, a system in which the urging capable of returning the external mount 3 to the raised position is generated by the expansion of a gas (or alternatively a liquid), while the pivoting of the external mount 3 towards the closed position is opposed by the resistant force generated by the compression of that gas (or liquid).

The invention according to this variant enables the lens to be interchanged in a manner which is simple and quick for the user, thus permitting the replacement of the lens in the case of damage or rendering possible the use of lenses of different colours, or also permitting easy removal of the lens in order to clean it.

The invention also has the advantage of permitting rapid access to the inside of the mask, when it is being worn, in order in particular to enable any spectacles to be put on, to enable their position on the face to be adjusted or to enable the lenses thereof to be cleaned.

Yet another advantage resides in the fact that the ability to pivot the external mount open and to remove the lens enables the exchange of air inside the mask to be increased, which brings about a consequent greater transpiration of the user's face, which is particularly important in the case of excessive sweating. This function is also ensured without requiring the user to take the mask off his face.

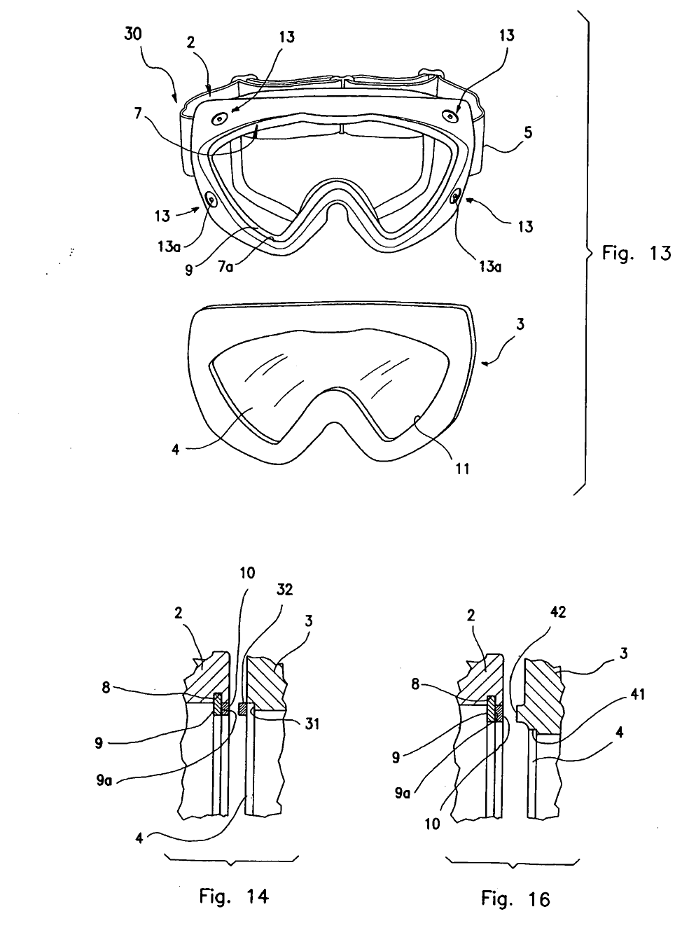

Referring to FIGS. 13 and 14, 30 generally indicates a third embodiment of mask-form goggles according to the invention in which components analogous to those of the first embodiment described, in relation to the mask 1, are indicated by the same reference numerals.

The mask-form goggles 30 differ from the mask-form goggles 1 principally in that the lens 4 is connected permanently to the external mount. For that purpose, it is provided that a seat 31 suitable for accommodating and holding rigidly the corresponding peripheral edge of the lens, for example by interlocking or by adhesive bonding or another form of mechanical securing, is formed in the mount 3 along the peripheral profile of the opening 11. 32 indicates a soft seal element (produced in material corresponding to that of the seal 10) fitted along the peripheral profile of the lens in a position facing the seal 10 provided on the bezel 9 of the internal mount. After the internal mount has been locked onto the external mount by means of the press-studs 13, the mutual contact between the seals 10 and 32 ensures sealing between the mounts which are coupled to each other.

The invention according to this variant therefore enables the lens-carrying external mount to be interchanged, thus permitting the replacement of the mount by other mounts, for example, having lenses of a different colour.

A further advantage is that of permitting free access to the inside of the mask, when it is being worn, in order in particular to enable any spectacles to be put on rapidly, to enable their position on the face to be adjusted or to enable the corresponding lenses thereof to be cleaned.

The free and complete access to the inside of the mask also advantageously facilitates cleaning operations.

Referring to FIGS. 15 and 16, 40 indicates a fourth embodiment of mask-form goggles according to the invention in which components analogous to those of the second embodiment described, in relation to the mask 20, are marked with the same reference numerals.

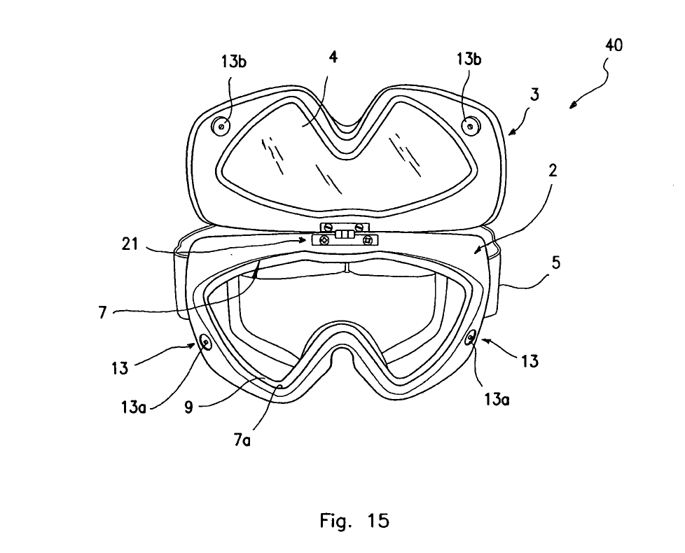

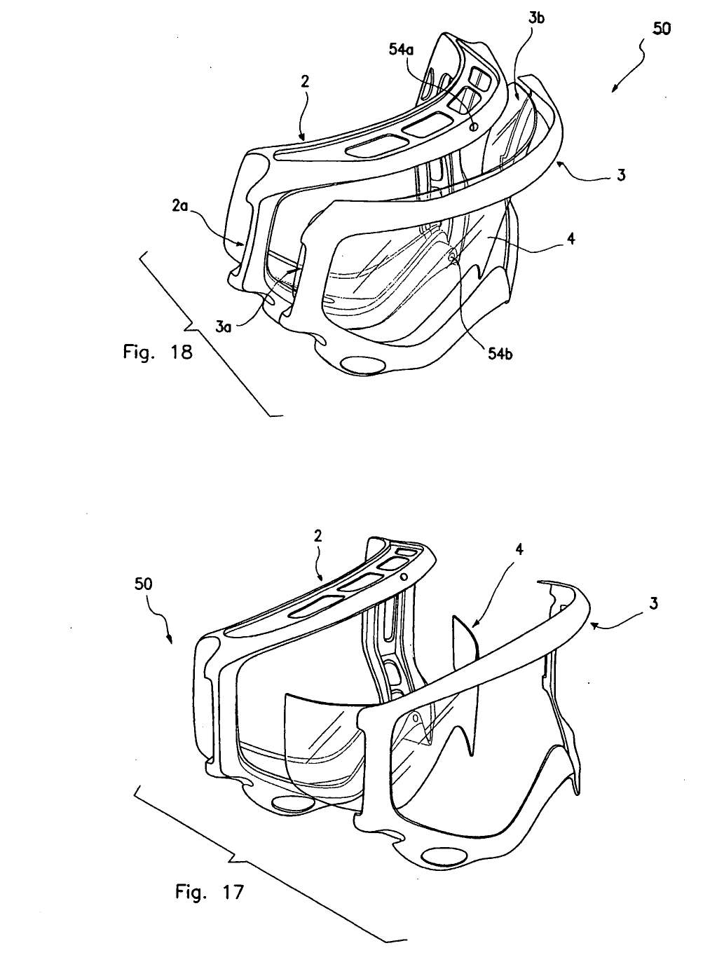

The mask-form goggles 40 differ from the mask-form goggles 20 owing principally to the fact that the lens 4 is connected permanently to the external mount 3. For that purpose, it is provided that a seat 41 suitable for accommodating and holding rigidly the corresponding peripheral edge of the lens, for example by interlocking or by adhesive bonding or another form of mechanical securing, is formed in the mount 3 along the peripheral profile of the opening 11. 42 indicates a projection which protrudes from the internal surface of the mount 3, that is to say, the surface facing the internal mount 2, and which extends along the peripheral edge of the front opening 11. The projection 42 is provided in a position corresponding to and facing the seal 10 provided on the bezel 9 of the internal mount 2 (FIG. 16). When the external mount has been locked onto the internal mount by the pair of press-studs 13 (as provided in the mask 20), the mutual contact between the seal 10 and the projection 42 ensures sealing between the mounts coupled to each other.

A principal advantage of this further variant of the invention is that it permits free access to the inside of the mask (by pivoting the internal mount as far as the raised position of complete opening), when it is being worn, in order, in particular, to enable any spectacles to be put on rapidly, to enable their position on the face to be adjusted or to enable the corresponding lenses thereof to be cleaned.

The free and complete access to the inside of the mask also advantageously facilitates the operations of cleaning the lens surfaces, enabling any condensation formed on the inside of the lens to be rapidly removed.

Yet another advantage resides in the fact that the ability to pivot the lens-carrying external mount open enables the exchange of air inside the mask to be increased, which brings about a consequent greater transpiration of the user's face, which is particularly important in the case of excessive sweating and also enables the misting-up of the lens to be opposed.

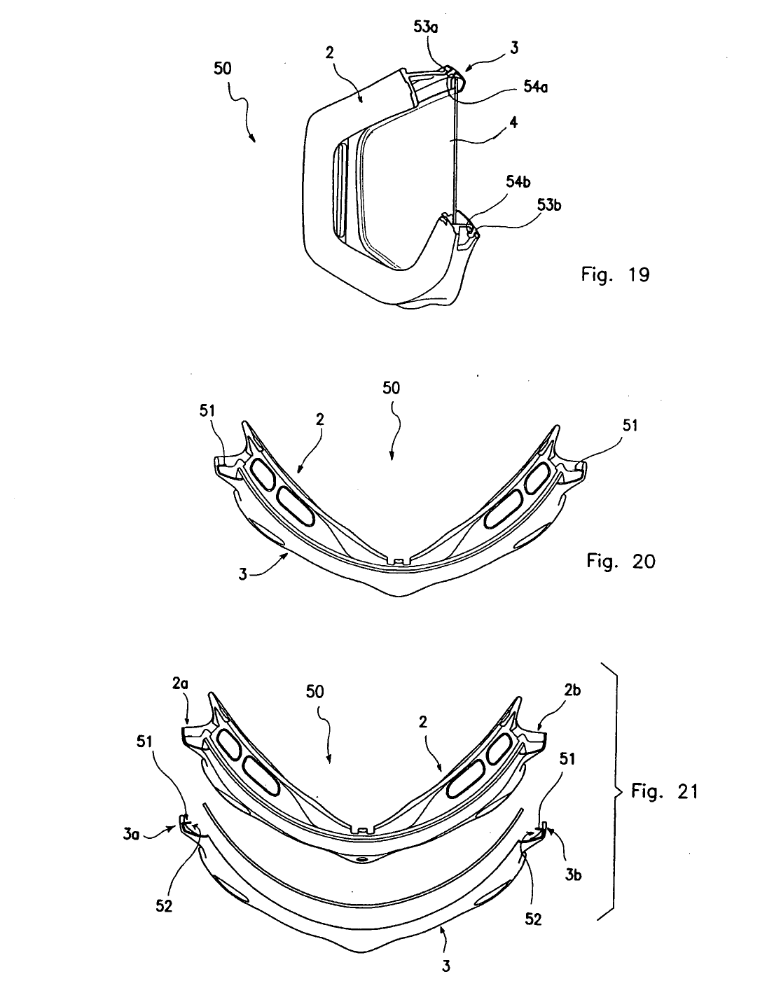

Referring to FIGS. 17 to 21, 50 indicates a fifth embodiment of the invention in which components analogous to those of the third embodiment described, in relation to the mask-form goggles 30, are marked by the same reference numerals.

The mask-form goggles 50 differ from the mask-form goggles 30 principally owing to the fact that the releasable coupling between the internal mount and the external mount is obtained by coupling between surface portions of the respective mounts, in particular by interlocking as specified in greater detail hereinafter.

For that purpose, the external mount 3 has an overall structure with reduced transverse thickness so that it is resiliently flexible, having a flexibility which is, overall, greater than that of the internal mount 2. Identified in the opposite lateral portions of the external mount, indicated 3a, 3b, are corresponding mount edges having projecting appendages 51 such as to define respective coupling profiles 52 capable of engaging the corresponding lateral portions 2a, 2b of the internal mount 2 for interlocking between the mounts. It will be appreciated that there is a substantial form-fit between the surfaces of the profiles in engagement with one another. This fit is obtained by an initial resilient deformation of the external mount 3, which is intended to move the opposite appendages 51 of the external mount away from each other in order then to enter into coupled engagement with the edges of the internal mount, ensuring firm coupling between the mounts after the resilient return of the external mount.

It is also provided that the mounts 2, 3 are equipped with additional means and counter-means for releasable coupling positioned along portions of the mounts at the location of the upper eyebrow arch and on the opposite lower front portion. Those means comprise appendages or protuberances projecting from the external mount and capable of entering into coupling engagement with respective seats or cavities formed in the internal mount in a corresponding position. Preferably, a first protuberance 53a is provided which has a rectangular cross-section with rounded corners and which has a widened head at its free end. The protuberance 53a projects from the mount 3 in a central position of the upper eyebrow arch portion thereof and is arranged to engage a cavity 54a which is formed in the internal mount and which has a slot-form opening with a cross-section such as to ensure interlocking when the protuberance has been pressed into the cavity.

A second protuberance 53b also extends centrally on the lower portion of the external mount 3, has a structure analogous to the first protuberance and is capable of entering into coupling engagement with a respective cavity 54b formed in the internal mount in a corresponding position. The coupling between the mounts ensured by the protuberances 53a, 53b has the function of temporary securing during the coupling of the mounts and is also used to oppose the deformation of the external mount about a vertical axis during the assembly of the mounts.

The lens 4 is secured permanently to the external mount 3 without any possibility of being separated from the external mount by the user. Thus, the lens-carrying external mount (together with the lens) can advantageously be replaced by other corresponding mounts, for example, having different colours or combined with lenses of a different colour, in order to guarantee the user a wider range of possible combinations. The invention according to this variant thus enables the lens-carrying external mount to be interchanged. A further advantage is that of permitting free access to the inside of the mask, when it is being worn, in order in particular to enable any spectacles to be put on quickly, to enable their position on the face to be adjusted or to enable the corresponding lenses thereof to be cleaned.

The free and complete access to the inside of the mask also advantageously facilitates cleaning operations. Yet another advantage resides in the fact that the ability to remove the external mount and the lens enables the exchange of air inside the mask to be increased, which brings about a consequent greater transpiration of the user's face, which is particularly important in the case of excessive sweating. This function is also ensured without requiring the user to take the mask off his face.

The invention thus achieves the proposed objects, obtaining the above-mentioned advantages over the known solutions.

1. Mask-form goggles, particularly for sporting use, comprising a first front mount and a second juxtaposed mount and also a lens held on at least one of the mounts, the first and the second mounts extending in such a manner that they surround the lens, and means for releasable mutual coupling are provided between the first and the second mount and are such as to permit access to the inside of the mask, when it is being worn, by moving one of the mounts away from the other.

2. The mask-form goggles according to claim 1, wherein the first mount is arranged to be held in position placed against the face, when the mask is being worn, and comprises a complete front opening at the location of the lens such as to permit access to the region of the face protected by the lens, when the mask is being worn, by moving the second mount away.

3. The mask-form goggles according to claim 1, wherein the lens is secured to the second mount.

4. The mask-form goggles according to claim 1, wherein the lens is held releasably between the mounts in such a manner as to ensure the interchangeability of the lens with respect to the mounts.

5. The mask-form goggles according to claim 1, also comprising hinge means between the mounts for hinging the second mount to the first mount.

6. The mask-form goggles according to claim 5, comprising resilient means which act between the mounts in order to urge the second mount away from the first mount, by pivoting about a hinge axis (X).

7. The mask-form goggles according to claim 1, wherein the releasable coupling means comprise means and counter-means for snap-locking by pressure which are provided on the first mount and the second mount, respectively.

8. The mask-form goggles according to claim 7, wherein the snap-locking means and counter-means comprise at least one press-stud acting between the mounts.

9. The mask-form goggles according to claim 1, wherein the releasable coupling means comprise coupling means between corresponding surface profiles of the first and second mounts.

10. The mask-form goggles according to claim 4, comprising a bezel element which projects from the front opening of the first mount and on which is defined a locating surface for abutment with the lens.

11. The mask-form goggles according to claim 10, wherein a first sealing element is interposed between the bezel element and the lens and is preferably secured to the bezel.

12. The mask-form goggles according to claim 11, wherein a respective second sealing element is interposed between the lens and the second mount in such a manner that the lens is held between the mounts, with the releasable coupling means, with sealing between the mounts.

13. The mask-form goggles according to claim 8, wherein the at least one press-stud is at least partially nested in the corresponding mount in order to limit or at most eliminate portions of the stud projecting from the surface profile of the mounts.

14. The mask-form goggles according to claim 5, wherein the hinge means comprise a first hinge element and a second hinge element which are associated with the first mount and the second mount, respectively, and which are articulated to each other about a hinge axis at the location of a central region of eyebrow arch portions of the mounts.

15. The mask-form goggles according to claim 6, wherein the resilient means comprise at least one torsion spring having opposite ends which act on the first mount and on the second mount, respectively.

16. The mask-form goggles according to claim 15, wherein the spring is a helical spring arranged coaxially with a hinge axis (X).

17. The mask-form goggles according to claim 9, wherein the coupling means comprise appendages extending from the second mount and defining coupling profiles capable of engaging corresponding portions of the first mount for interlocking between the mounts.

18. The mask-form goggles according to claim 17, wherein the coupling means are provided along opposite lateral edges of the mounts.

19. The mask-form goggles according to claim 17, wherein the second mount has an overall resilient compliance greater than that of the first mount, so that the appendages are coupled to the corresponding profiles of the first mount by deformation and resilient return of the second mount.

20. The mask-form goggles according to claim 9, wherein the releasable coupling means also comprise at least one protuberance which extends from one of the mounts in the direction towards the other and which is capable of engaging a respective coupling seat on the other mount.

21. The mask-form goggles according to claim 20, wherein the second mount is provided with a pair of protuberances which extend centrally from each respective front portion of the second mount and which are arranged to engage respective coupling seats arranged in a correspondingly facing position on the first mount.

22. The mask-form goggles according to claim 1, wherein the first mount is associated with a seal for sealing bearing contact with a user's face, and a connecting structure having superior flexibility characteristics to those of the first mount being provided between the first mount and the seal so that any resilient deformation induced in the first mount by the coupling to the second mount is compensated for at least partially by the connecting structure.

23. The mask-form goggles according to claim 22, wherein the connecting structure is constructed in the form of a bellows or accordion element with predominant resilient compliance in predetermined directions.

24. The mask-form goggles according to claim 1, wherein the releasable coupling means comprise slide means supported movably on the second mount and capable of interlocking with respective cavities provided in the first mount in a corresponding position, so that the slide mean are movable between a position of interlocking with the respective cavity, in order to lock the mounts to each other, and a position of disengagement with respect to the cavity, in order to uncouple the mounts from each other.
